(19) United States
(12) Patent Application Publication
(10) Pub. No.: US 2024/0123136 A1
(43) Pub. Date: Apr. 18, 2024

(54) MULTI-COMPONENT COMMUNICATION SYSTEM FOR INFUSION LINE IDENTIFICATION

(71) Applicant: ORION INNOVATIONS INC., St. George, UT (US)

(72) Inventor: Wayne A. PROVOST, St. George, UT (US)

(21) Appl. No.: 18/381,567

(22) Filed: Oct. 18, 2023

Related U.S. Application Data
(60) Provisional application No. 63/417,280, filed on Oct. 18, 2022.

(51) Int. Cl. A61M 5/14

(52) U.S. Cl. CPC A61M 5/14

(57) ABSTRACT

Disclosed is an infusion line indication system that includes one or more indicator devices configured for attachment to a medical line such as an infusion line, and a dongle that is connectable to a line control unit such as an infusion line pump. The one or more indicator devices are configured to provide an indicator signal such as a light signal. The dongle and the one or more indicator devices are capable of being wirelessly connected. The dongle is configured to transmit an indicator instruction to the one or more indicator devices in response to one or more line parameters sensed by sensors of the indicator devices or by the line control unit to thereby cause the indicator signal to change.

### CROSS-REFERENCE TO RELATED APPLICATIONS

This application claims priority to and the benefit of U.S. Provisional Patent Application No. 63/417,280, filed Oct. 18, 2022 and titled “MULTI-COMPONENT COMMUNICATION SYSTEM FOR INFUSION LINE IDENTIFICATION,” the entirety of which is incorporated herein by reference.

### BACKGROUND

### Technical Field

This disclosure relates to systems and devices configured to enable identification of a medical line, such as an infusion line, and/or sensing of various parameters of the medical line.

### Related Technology

In certain medical settings such as hospital settings, patients are often administered therapeutic fluids via infusion lines, also typically referred to as intravenous (IV) lines. Infusion lines generally consist of flexible, polymer tubing connected at one end to a fluid source and at another end to a needle or port that provides access to a vessel of a patient. It is not uncommon for multiple infusion lines, each connected to a different source of fluid, to be used simultaneously to deliver several therapeutic fluids at once to a single patient. It is also not uncommon for the needles or ports to be located adjacent to one another, such as multiple adjacent needles providing access into the brachial vein running through the arm of the patient.

While the simultaneous use of multiple infusion lines can provide numerous benefits, challenges can also be encountered. For instance, when multiple infusion lines are used to administer multiple therapeutic fluids to a single patient, it can become cumbersome and difficult to readily identify one line from another. Thus, it can be difficult to quickly and accurately identify a particular therapeutic fluid source and corresponding therapeutic fluid output compared to another medication source and its corresponding therapeutic fluid output. This problem is aggravated by the tendency of each of the lines to coil up to their packaged configuration and consequently tangle with other lines or tangle under bed sheets or clothing.

Quick identification of a particular therapeutic fluid source is often required in emergency situations. For example, when a patient hooked up to multiple lines is in need of emergency intravenous administration of a therapeutic fluid not currently being provided through one of the lines, it is necessary to immediately provide that therapeutic fluid. If a blood vessel cannot rapidly be located into which the therapeutic fluid can be injected, it is common practice to provide the drug through an infusion line in which a therapeutic fluid is already being administered. This practice of using existing infusion lines to administer new therapeutic fluids is also common in non-emergency situations.

The person administering the drug must be sure that the infusion line through which the new therapeutic fluid is administered is carrying a therapeutic fluid which is compatible with the new therapeutic fluid. Severe results may occur if a new therapeutic fluid is injected through an infusion line in which the therapeutic fluid already flowing therethrough is not compatible with the new therapeutic fluid. For example, if heparin is injected into an infusion line through which lidocaine is already flowing, a flakey precipitate will form in the mixture which can be dangerous to a patient. Similarly, mixing insulin with certain chemotherapy drugs in a common infusion line can be extremely dangerous for a patient.

In addition, medical personnel often desire to know the status of infusion line conditions, such as line pressure, fluid flow, whether the line is restricted, and whether there are bubbles or discontinuities in the line. Some infusion line pumps can sense certain conditions such as pressure within the line. However, as related to the identification problem discussed above, even if such information is provided by the pump, the user may still have difficulty identifying which line is associated with a particular measurement. For example, if an infusion line pump indicates that an infusion line has low pressure (and/or some other error type), the user must still identify the infusion line ends that belong to the infusion line which was measured by the pump. As discussed above, this can be difficult where there are multiple infusion lines running alongside each other and potentially wrapping, twisting, and/or coiling about each other. Moreover, there may be circumstances where the pump fails to provide an alarm altogether, in which case a potentially dangerous situation may go unnoticed and remedied.

As a result of the difficulties in distinguishing between multiple infusion lines and their associated fluid sources and outputs and the potentially life-threatening possibilities that can occur if incompatible therapeutic fluids are injected through the same infusion line, there is an ongoing need for devices that allow for ready and accurate identification of individual infusion lines with their associated fluid sources and outputs. In addition, there is an ongoing need for systems and devices capable of effective sensing of medical lines and effective communication/indication of sensed states (e.g., emergency situations). This need is prevalent both for systems with pumps and for systems without pumps, such as those that rely on conventional gravity-fed infusion.

### SUMMARY

Disclosed is a medical line indication system that includes one or more indicator devices configured for attachment to or integration with a medical line (e.g., an infusion line), and a dongle that is connectable to a line control unit. The line control unit can also be a pump, though in other embodiments, the line control unit is not a pump. The system can therefore beneficially function as a “pump replacement” that provides line sensing and communication/indication functions even in simple gravity bag applications. Such sensing functionality has conventionally only been available when using relatively complex pump assemblies. These complex assemblies are, of course, not available in many emergency situations or third-world countries, for example.

The dongle can be a conventional dongle that is physically separatable from the line control unit (e.g., via a hardware connection such as universal serial bus (USB)). Additionally, or alternatively, the dongle may be fully or partially integrated into the line control unit, such as where the line control unit is already capable of wireless connection to the indicator devices.

While most of the examples described herein discuss indicator devices that are selectively attachable to medical lines, it will be understood that the same principles readily apply to embodiments where the indicator devices are fully or partially integrated with the medical line. For example, indicator devices can be fully or partially integrated with a medical line during manufacture of the medical line.

The one or more indicator devices are configured to provide an indicator signal such as a light signal and/or audible signal. The dongle and the one or more indicator devices are capable of being wirelessly connected. The dongle is configured to transmit an indicator instruction to the one or more indicator devices. Additionally, or alternatively, the dongle can be configured to receive sensor data from one or more sensors associated with the one or more indicator devices. The system can thus modulate the one or more indicator devices in response to a medical line condition sensed by the line control unit and/or sensed by the one or more sensors of the indicator devices. For example, the line control unit and/or the sensors can detect a medical line condition, and the dongle can receive such data and, in response, send an indicator instruction to the indicator devices to thereby cause the indicator signal to change. Accordingly, the disclosed embodiments can beneficially display infusion status information at one or more locations along the infusion line.

As an example, the line control unit can include or be associated with a pump, and when the pump senses a low flow condition, the dongle can transmit a signal to the one or more indicator devices to cause the indicator signal to change (e.g., to cause the indicator devices to light up with a red warning light). Other medical line conditions sensed by the line control unit can be associated with different indicator signals. For example, the dongle can cause the one or more indicator devices to generate an off signal or a different colored light (e.g., green) when a normal flow condition is sensed.

Changes to the indicator signal may include changes in color and/or pattern of light and/or turning on or turning off of a light signal. In some embodiments, the system is configured to generate different indicator signals in response to different respective infusion conditions sensed by the line control unit. For example, a certain color and/or pattern may be associated with “good” conditions and another color and/or pattern may be associated with “bad” conditions. Additionally, or alternatively, a first set of colors and/or patterns may be associated with a first type of condition (e.g., pressure), a second, different set of colors and/or patterns may be associated with a second, different type of condition (e.g., concentration), and so on.

Some embodiments of indicator devices may additionally or alternatively utilize audio to provide indications. For example, an indicator device may emit certain sounds and/or sound patterns that correspond to medical line conditions sensed by the line control unit.

Example medical line conditions that can be measured by the sensors of the indicator devices and/or the line control unit and that can correspond to changes in the indicator signal(s) generated by the one or more indicator devices include line pressure, fluid flow rate, precipitate detection, gas concentration, medicine concentration, air bubble detection, temperature, other infusion conditions that can be sensed by an infusion pump or other line control unit as known in the art or as developed in the future, and combinations thereof.

The indicator devices can include any suitable sensor known in the art for detecting such parameters. For example, an indicator device can include a flow rate sensor (e.g., ultrasonic, optical, and/or mechanical (such as an impeller) sensors), an air bubble or precipitate sensor (e.g., optical, infrared, and/or ultrasonic sensor), a temperature sensor (e.g., infrared sensor and/or thermocouple), and/or a salinity sensor (e.g., optical, infrared, or ultrasonic sensor).

The line control unit can function to store sensor data and/or other indicator device data (e.g., usage data), to process received data, and to generate indicator instructions. The line control unit can also include its own indication capabilities (e.g., an acoustic alarm) separate from the indicator devices. In some embodiments, the line control unit can be configured to utilize artificial intelligence methods, including machine learning methods known in the art, to analyze received sensor data and/or generate indicator instructions.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates a conventional infusion line assembly;

FIGS. 2A and 2B illustrate infusion line assemblies incorporating an infusion line identification system comprising one or more indicator devices (e.g., light sources) and a pump dongle configured for communicative connection with a line control unit in the form of an infusion line pump;

FIGS. 3A and 3B illustrate embodiments of line indication systems that utilize an external computer device as a line control unit;

FIGS. 4A and 4B illustrate an example of a line indication system operating to sense an air bubble and activate an indicator signal in response;

FIGS. 5A and 5B illustrate an example of a line indication system operating to sense precipitate formation and activate an indicator signal in response;

FIG. 6 illustrates an example of a line indication system operating to sense and display various parameters of the infusion line; and

FIGS. 7A and 7B illustrate example pairing methods that can be used to pair the dongle to the indicator devices.

### DETAILED DESCRIPTION

### Introduction

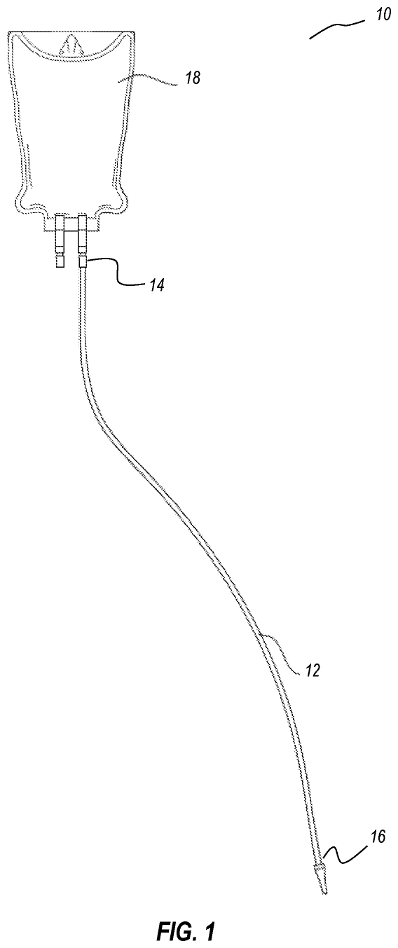

FIG. 1 illustrates a conventional infusion line assembly 10 including an infusion line 12 connected to a therapeutic fluid bag 18. The infusion line 12 includes a first end 14 and a second end 16. As shown, one end may be connected to the therapeutic fluid bag 18, leaving the other end free to be intravenously inserted into the patient. As discussed above, issues can arise in medical settings due to the difficulty in properly matching one end of an infusion line to its opposite end. That is, where multiple infusion lines are associated with a patient, it can often be difficult to readily identify which line at the patient end is attached to which therapeutic fluid.

U.S. Pat. No. 10,722,641, which is incorporated herein in its entirety by this reference, describes infusion line assemblies configured to enable identification of an infusion line via multiple indicator devices (as light sources) that are selectively attachable to an infusion line and that are communicatively coupled to one another via a wireless connection. The light sources are configured such that when a first light source is manually activated to provide a light signal, a second light source is also automatically activated, without requiring manual activation, to provide a corresponding light signal, and vice versa.

Such selectively attachable indicator devices beneficially enable users (e.g., medical personnel) to easily attach the devices to an infusion line, or multiple sets of such indicator devices to multiple respective infusion lines, in a conventional setting without the need for additional attachment equipment or custom manufacturing processes. Such indicator devices may be utilized in the embodiments described herein.

While infusion line therapy devices for administering therapeutic fluids are exemplified below, other embodiments may be implemented for other applications, both within the medical field and in other technical fields. Various components of the illustrative embodiments may be excluded or replaced with other components known and used in the art. By way of non-limiting example, some of the exemplary embodiments include therapeutic fluid bags, pumps, and connectors. Each of these components could be eliminated or replaced with other components. For instance, various types of pumps, or no pump at all, can be used with the disclosed embodiments. Similarly, various types of fluid sources and connectors other than conventional therapeutic fluid bags and Y-connectors could be employed.

Similarly, while the specific examples discussed herein relate to infusion lines, the skilled person will readily understand that the same components and principles may be applied to adaptors for other types of medical lines where sensing and/or identification may be beneficial. For example, embodiments described herein may be connected to other types of intravenous (IV) lines such as central lines (e.g., central venous catheter lines) and peripherally inserted central catheter (PICC) lines, nasogastric tubes, arterial lines, peritoneal dialysis lines, umbilical lines (typically used for newborns), long-term access catheters (e.g., Hickman or Broviac catheters), wound drainage lines, or any other medical line where integration with sensor and/or indicator functionality is beneficial.

### Communication System for Control Unit & Indicator Devices

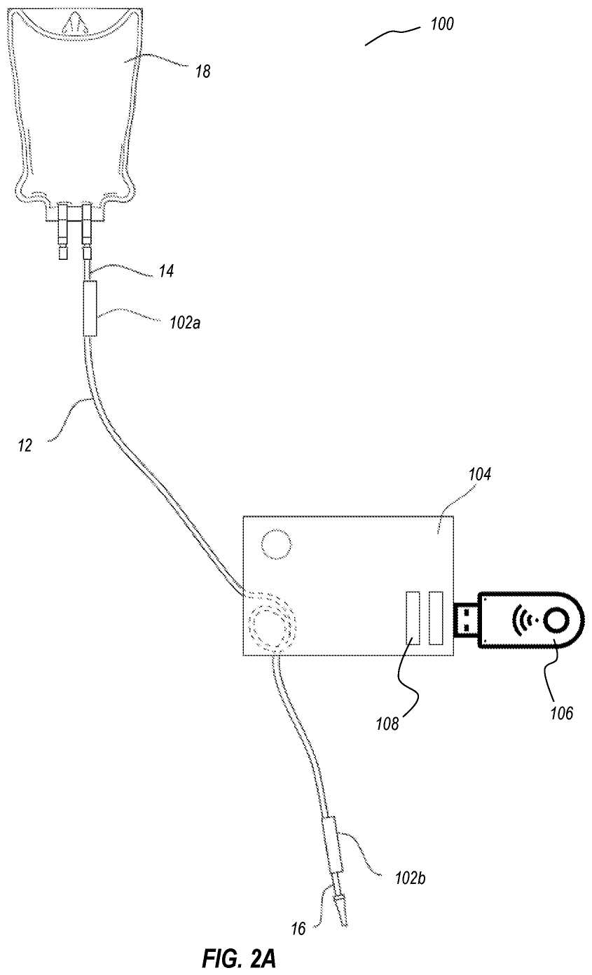

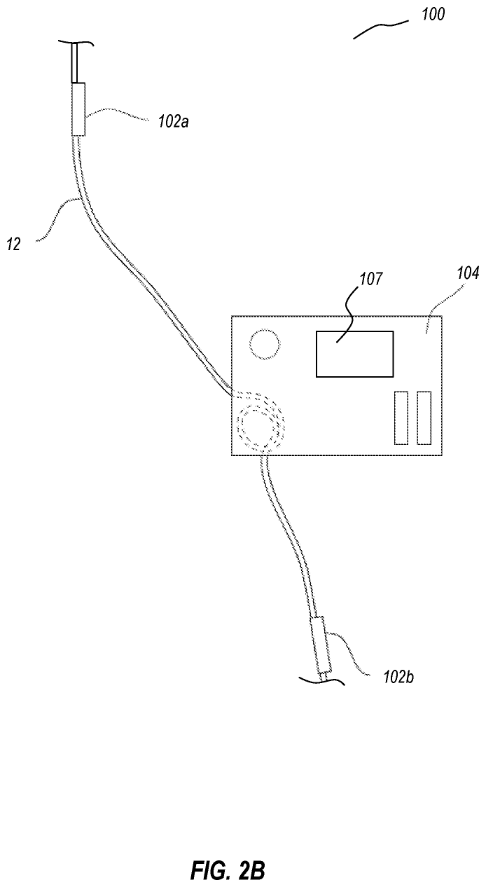

FIG. 2A illustrates an infusion line indication system 100 that may be implemented with an infusion line assembly such as shown in FIG. 1. The illustrated system 100 includes a set of indicator devices 102 (including indicator device 102a and indicator device 102b) attached to the infusion line 12. The illustrated indicator devices 102 are attached at or near the ends 14 and 16 of the infusion line 12. Other embodiments may include additional indicator devices and/or may position indicator devices in different positions along the infusion line 12. The indicator devices 102 can optionally include an anti-microbial coating to help minimize the risk of spreading infection.

The illustrated system 100 also includes a line control unit, which in this embodiment is represented as an infusion line pump 104. The pump 104 may be, for example, a rotary pump, peristaltic pump, or other type of pump suitable for an infusion line application. As described in greater detail below, the line control unit need not be associated with a pump, and may additionally or alternatively include an external computer device, for example.

The system 100 also includes a dongle 106 that can be connected to the pump 104 at a hardware connection location 108. The dongle 106 may be connected to the pump 104 via a universal serial bus (USB) connection, for example, though other hardware connections and/or suitable wireless connections known in the art may additionally or alternatively be utilized. The dongle 106 may include a controller, a communication module for communicating with the pump 104, and a wireless transmitter and/or wireless receiver for communicating with the indicator devices 102a and 102b. The communication module may include an application programming interface (API), for example, suitable for enabling communication with the pump 104.

The dongle 106 is capable of wireless connection with the indicator devices 102. The wireless connection can include, for example, an ultra high frequency (UHF) radio wave connection, such as Bluetooth®. The dongle 106 is thereby capable of receiving sensor data from the indicator devices 102 and/or sending an indicator instruction to the indicator devices 102. The indicator instruction can be in response to one or more infusion conditions sensed by the infusion line pump 104 and/or by sensors of the indicator devices 102 and can operate to cause the indicator signal to change.

As used herein, an “indicator signal” is the signal generated by the one or more indicator devices 102 and may include light and/or audio. As used herein, an “indictor instruction” is an instruction sent by the dongle 106 to the one or more indicator devices 102 that causes a change in the indicator signal. The indicator signal may change by turning on or off, by changing the light color, by changing the light brightness or intensity, by changing the light pattern (e.g., non-flashing vs. flashing), and/or by changing audio volume, pitch, or tempo, for example.

Where a set of multiple indicator devices are used, the indicator signals may be synchronized among the multiple indicator devices so that they each provide the same indicator signal at the same time. Each set of indicator devices may include, for example, two or three indicator devices, though more indicator devices could also be provided. A line indication system may be configured so that one line control unit controls one set of indicator devices. Alternatively, in some embodiments, a line control unit can be configured to control multiple different sets of indicator devices. For example, multiple sets of indicator devices may be associated with different respective medical lines, either on the same patient or across multiple patients. The line control unit can be configured to independently control each different set. As an example, in a triage and/or military scene where multiple patients are in relatively close proximity, a single line control unit may be utilized to control different indicator device sets each associated with a different patient.

Example infusion conditions that can be measured by the sensors of the indicator devices and/or the line control unit (e.g., the infusion pump 104) and that can correspond to changes in the indicator signal generated by the one or more indicator devices 102 include line pressure, concentration of one or more fluid components (e.g., salinity, gas, medicine), air bubble detection, fluid temperature, other infusion conditions that can be sensed by an infusion pump as known in the art or as developed in the future, and combinations thereof.

As mentioned above, some embodiments are configured for two-way communication in which the dongle 106 is configured to receive data from the one or more indicator devices 102 and to communicate the received data with the infusion line pump 104 and/or other line control unit. For example, an indicator device 102 can sense that a medicine has been injected into the infusion line 12 and can send a corresponding indication to the dongle 106. The dongle 106 can then store this in its own memory and/or communicate the receipt of such an indication to the pump 104 for storage in memory associated with the pump 104.

In some embodiments, the one or more indicator devices 102 include one or more sensors. Such sensors may include those disclosed herein for the pump 104, such as pressure sensors, flow sensors, concentration sensors (e.g., medicine, salinity), bubble/discontinuity sensors, precipitate sensors, temperature sensors, and the like.

FIG. 2B illustrates an alternative configuration of the system 100 in which the functions of the dongle 106 are carried out by one or more integral components 107 of the pump 104. For example, the pump 104 may be capable of directly communicating with the indicator devices 102 and associated sensors without the need for a separate dongle. As used herein, the an “integrated dongle” or a dongle that “is integrated with the line control unit” refers to embodiments where the line control unit includes one or more integrated components capable of carrying out the communication and processing functions described above for the dongle 106.

In some embodiments, a dongle 106 is utilized primarily for sending and receiving data, while the pump 104 and/or other line control unit are primarily utilized for processing of sensor data and generation of indicator instructions. Accordingly, processing and communication operations can be divided among the dongle and one or more line control units in a variety of ways.

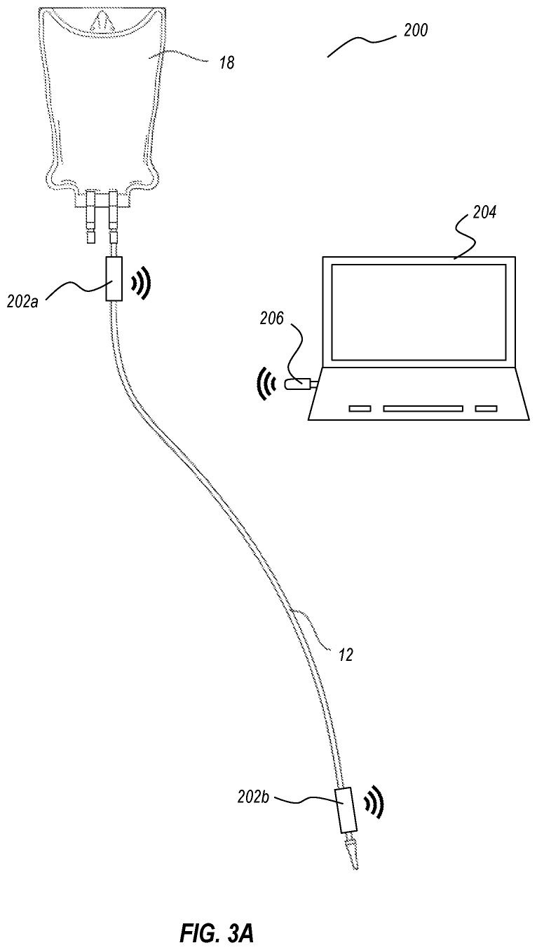

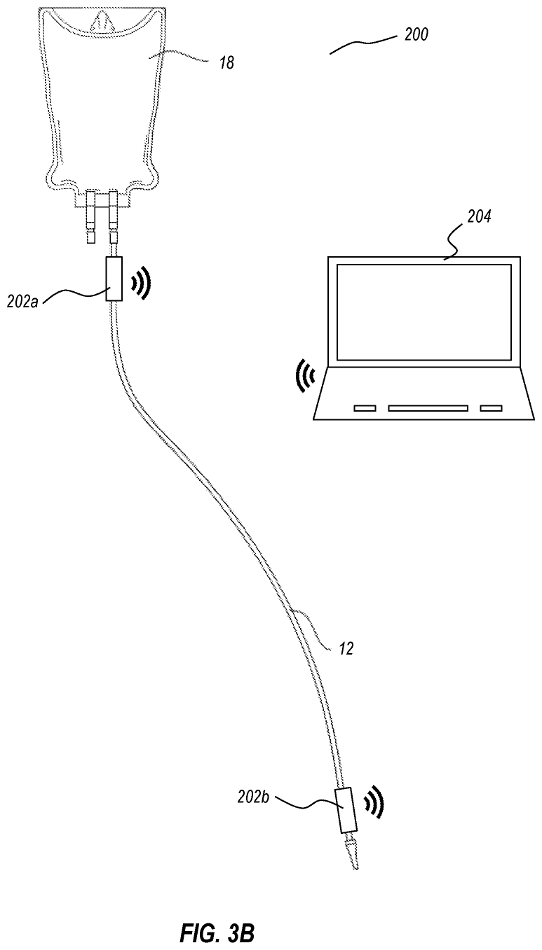

FIG. 3A illustrates an embodiment of a line indication system 200 that utilizes an external computer device 204 as a line control unit (rather than a pump). The external computer device 204 may include a dedicated/custom controller, laptop computer, desktop computer, mobile device (e.g., tablet, phone), other computer device, or combination thereof.

The system 200 includes a dongle 206 configured for wireless communication with the indicator devices 202. The system 200 may function similar to the line indication system 100, and the relevant description above is therefore applicable here as well.

FIG. 3B illustrates an alternative configuration of line identification system 200 in which the dongle 206 is omitted. The external computer device 204 instead includes an “integrated dongle” and thus includes one or more internal components that communicate with the indicator devices 202.

The inclusion of sensors in the indicator devices allows the system to provide sensing functionality (e.g., air bubble detection, precipitate detection, fluid flow measuring, and/or other sensing functions disclosed herein) without requiring a complex pump assembly or any pump assembly at all. For example, a system can include indicator devices with sensors and a non-pump line control unit. The line control unit in such applications can be a dedicated controller or a standard computer device (e.g., laptop, desktop, tablet, phone) with an application that manages communication with and control of the indicator devices. This allows implementation of the system with gravity-fed applications, which may be more common in field situations, emergency situations, or third-world country applications.

### Sensor Operation & Indicator Activation

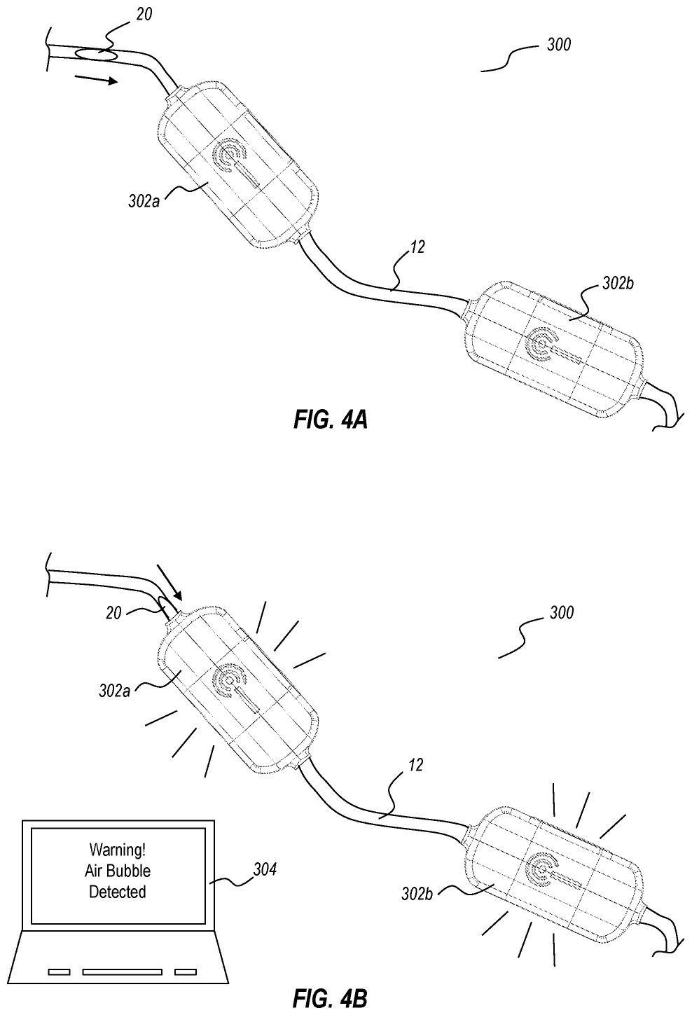

FIGS. 4A and 4B illustrate an example of a line indication system 300 operating to sense an air bubble and activate an indicator signal in response. The system 300 can include any combination of the features described in relation to line indication systems 100 or 200, and vice versa.

As shown in FIG. 4A, an air bubble 20 is disposed within the infusion line 12. As the air bubble 20 moves downstream through the infusion line 12, it eventually reaches an indicator device 302a. As shown in FIG. 4B, the indicator device 302a senses the air bubble 20 once it passes into or moves past the indicator device 302a. The sensor signal is communicated to the external computer device 304 (e.g., directly or via an external dongle intermediary) and subsequently receives an indicator instruction that causes the indicator devices 302 to activate (e.g., to turn on lights).

Note that even though the air bubble 20 was sensed by the indicator device 302a, the system 300 can operate to simultaneously activate both indicator devices 302. As shown, the external computer device 304 can also provide an indicator message (audio and/or visual display) corresponding to the indicator signal of the indicator devices 302.

The use of multiple indicator devices 302 on the line can enable additional benefits. For example, as the air bubble 20 is detected in the upstream indicator device 302a, the system 300 can first issue a warning indicator. If the air bubble 20 continues to be detected by downstream indicator devices as it moves toward the patient, the system 300 can progressively escalate the warning. The time elapsed between detection at the multiple indicator devices can also be utilized to calculate system flow rate (i.e., given a known inner diameter of the line).

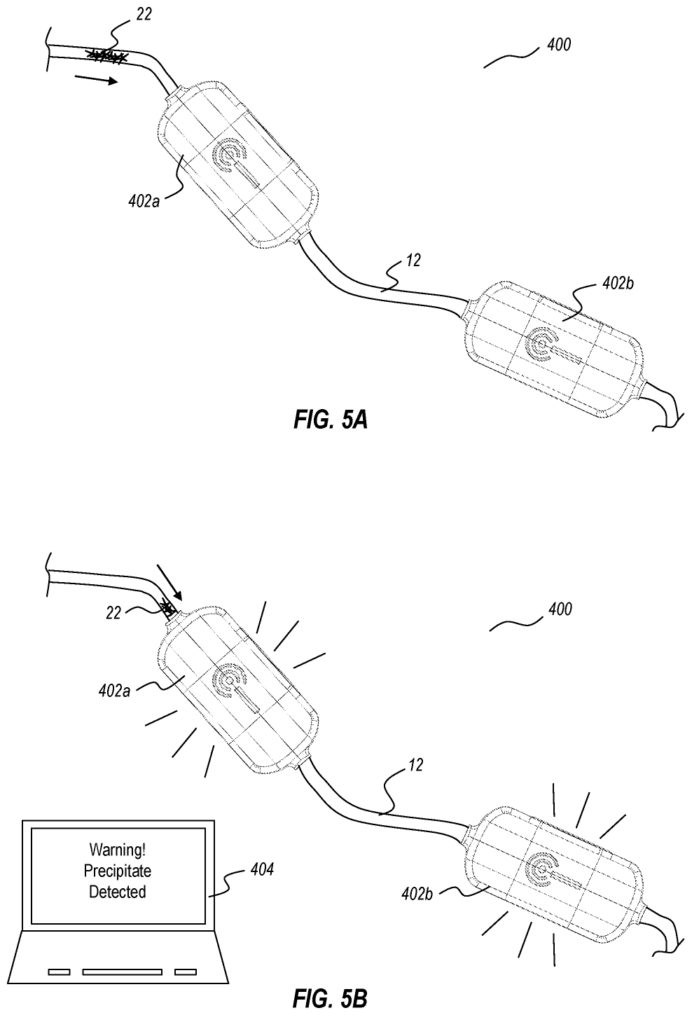

FIGS. 5A and 5B illustrate an example of a line indication system 400 operating to sense precipitate formation and activate an indicator signal in response. The system 400 can include any combination of the features described in relation to line indication systems 100 or 200, and vice versa.

As shown in FIG. 5A, precipitate 22 is disposed within the infusion line 12. As the precipitate 22 moves downstream through the infusion line 12, it eventually reaches an indicator device 402a. As shown in FIG. 5B, the indicator device 402a senses the precipitate 22 once it passes into or moves past the indicator device 402a. The sensor signal is communicated to the external computer device 404 (e.g., directly or via an external dongle intermediary) and subsequently receives an indicator instruction that causes the indicator devices 402 to activate (e.g., turn on lights).

Note that even though the precipitate 22 was sensed by the indicator device 402a, the system 400 can operate to simultaneously activate both indicator devices 402. As shown, the external computer device 404 can also provide an indicator message (audio and/or visual) corresponding to the indicator signal of the indicator devices 402.

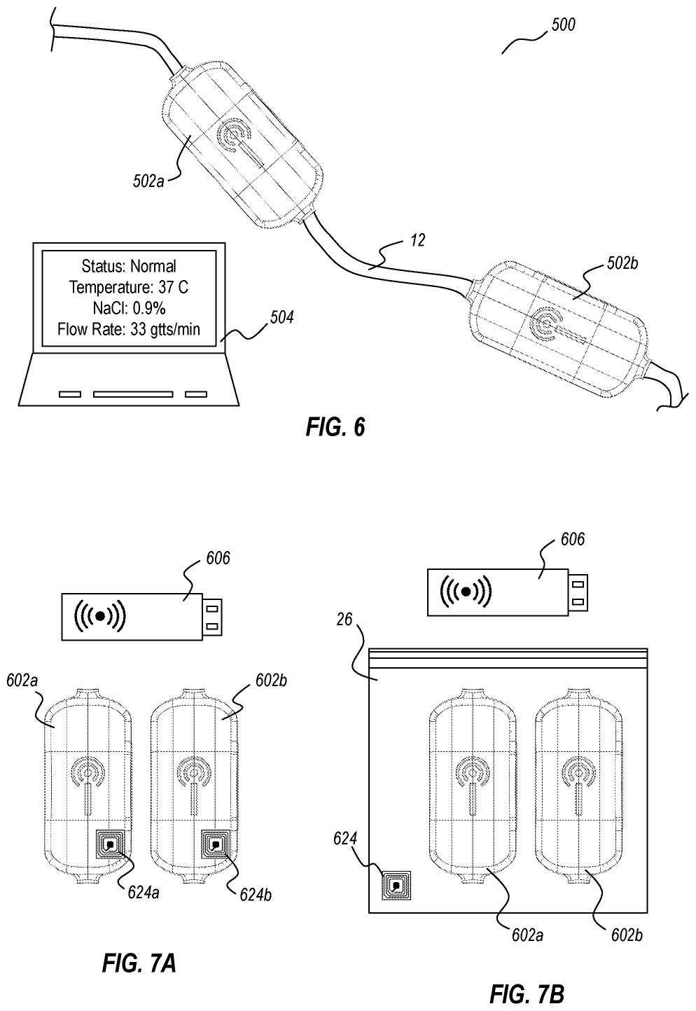

FIG. 6 illustrates an example of a line indication system 500 operating to sense various parameters of the infusion line 12. The system 500 can include any combination of the features described in relation to line indication systems 100 or 200, and vice versa. The indicator devices 502 can include various sensors as disclosed herein. Sensor data can be communicated to the external computer device 504 (e.g., directly or via an external dongle intermediary) where it can be stored and/or processed.

In some embodiments, one or more of the indicator devices can include a display screen for showing line parameter information such as flow rate and/or any of the other parameters disclosed herein. This can be in addition to or as an alternative to other displays such as those on an external computer device functioning as a line control unit.

### Indicator Device Pairing

FIGS. 7A and 7B illustrate example pairing methods that can be used to pair the dongle 606 to the indicator devices 602. In some implementations, the indicator devices 602 are single use, disposable components. In such cases, each new set of indicator devices 602 will need to be paired to the dongle 606.

The pairing process may utilize radio frequency identification (RFID) functionality. For example, as shown in FIG. 7A, the dongle can include an RFID reader and each indicator device 602 can include its own RFID tag 624 (i.e., indicator device 602a includes RFID tag 624a and indicator device 602b includes RFID tag 624b). The user can bring the indicator devices 602 into sufficient proximity to the dongle 606 to initiate pairing and establishment of the wireless connection.

Alternatively, as shown in FIG. 7B, the RFID tag 624 can be associated with the packaging 26 of the indicator devices 602. The user can thus initiate pairing and establish the wireless connection by bringing the packaging 26 into proximity with the dongle 606.

### Additional Computer/Controller Component Details

The indicator devices, dongles, and/or external computer devices described herein may be configured to include one or more executable components to form communicative links between the devices. The term “executable component” is the name for a structure that is well understood to one of ordinary skill in the art in the field of computing as being a structure that can be software, hardware, or a combination thereof.

For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media. The structure of the executable component exists on a computer-readable medium in such a form that it is operable, when executed by one or more processors of the computing system, to cause the computing system to perform one or more functions, such as the functions and methods described herein. Such a structure may be computer-readable directly by a processor—as is the case if the executable component were binary. Alternatively, the structure may be structured to be interpretable and/or compiled—whether in a single stage or in multiple stages—so as to generate such binary that is directly interpretable by a processor.

The term “executable component” is also well understood by one of ordinary skill as including structures that are implemented exclusively or near-exclusively in hardware logic components, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination thereof.

The terms “component,” “service,” “engine,” “module,” “control,” “generator,” or the like may also be used in this description. As used in this description and in this case, these terms—whether expressed with or without a modifying clause—are also intended to be synonymous with the term “executable component” and thus also have a structure that is well understood by those of ordinary skill in the art of computing.

The indicator devices pump dongles, and/or external computer devices described herein may also include one or more controllers/processors and one or more computer-readable storage media. Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium that can be used to store desired program code in the form of computer-executable instructions or data structures and that can be accessed and executed by a general purpose or special purpose computing system to implement the disclosed functionality of the invention. For example, computer-executable instructions may be embodied on one or more computer-readable storage media to form a computer program product. For the absence of doubt, such computer-readable storage media can also be termed “hardware storage devices,” which are physical storage media—not transmission media.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

Reference numerals referring to multiple instances of the same element may take the form of a reference numeral followed by a letter. For example, if component 150 is included multiple times in an embodiment, a general reference to such components and/or a reference to the collective set of such components may be referred to as “component 150,” whereas individual instances of the component may be referred to as “component 150a, component 150b, component 150c,” etcetera.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A medical line indication system, comprising:
one or more indicator devices configured for attachment to a medical line, the one or more indicator devices each being configured to provide an indicator signal; and
a dongle configured for communicative connection with the one or more indicator devices and with a line control unit,
wherein the dongle is wirelessly connectable to the one or more indicator devices, and
wherein the dongle is configured to transmit an indicator instruction to the one or more indicator devices in response to one or more line conditions sensed by the line control unit to thereby cause the indicator signal to change.

2. The system of claim 1, wherein the system comprises multiple indicator devices, wherein the indicator signal is synchronized among the multiple indicator devices.

3. The system of claim 1, wherein indicator signal includes a light.

4. The system of claim 1, wherein the one or more line conditions include line pressure, fluid flow rate, fluid temperature, bubble or precipitate detection, or concentration of a fluid component.

5. The system of claim 1, wherein the one or more indicator devices and the dongle are wirelessly connectable via an ultra high frequency (UHF) radio wave connection.

6. The system of claim 1, wherein the system is configured to generate different indicator signals in response to different respective infusion conditions sensed by the line control unit.

7. The system of claim 6, wherein the different indicator signals include different patterns and/or different colors of light.

8. The system of claim 1, wherein the one or more indicator devices include one or more sensors for sensing a line condition, and wherein the dongle is further configured to receive data from the one or more indicator devices and to communicate the received data with the line control unit.

9. The system of claim 8, wherein the data from the one or more indicator devices includes an indication that a medicine has been injected into the medical line.

10. The system of claim 1, wherein the line control unit is associated with a pump.

11. The system of claim 1, wherein the dongle is integrated with the line control unit.

12. A medical line indication system, comprising:
one or more indicator devices configured for attachment to a medical line, the one or more indicator devices each being configured to provide an indicator signal and each including one or more sensors for sensing a line condition;
a line control unit; and
a dongle configured for communicative connection with the one or more indicator devices and with a line control unit,
wherein the dongle is wirelessly connectable to the one or more indicator devices, and
wherein the dongle is configured to receive sensor data transmitted from the one or more indicator devices and to send an indicator instruction to the one or more indicator devices in response to thereby cause the indicator signal to change.

13. The system of claim 12, wherein the dongle is integrated with the line control unit.

14. The system of claim 12, wherein the system comprises multiple indicator devices, wherein the indicator signal is synchronized among the multiple indicator devices.

15. The system of claim 12, wherein indicator signal includes a light.

16. The system of claim 14, wherein the one or more line conditions include line pressure, fluid flow rate, fluid temperature, bubble or precipitate detection, or concentration of a fluid component.

17. The system of claim 12, wherein the one or more indicator devices and the dongle are wirelessly connectable via an ultra high frequency (UHF) radio wave connection.

18. The system of claim 12, wherein the system is configured to generate different indicator signals in response to different respective infusion conditions sensed by the line control unit.

19. The system of claim 12, wherein the line control unit is not a pump.

20. The system of claim 19, wherein the medical line is an infusion line and wherein the system is configured to use gravity feed for infusion.
